(19) United States
(12) Patent Application Publication
(10) Pub. No.: US 2008/0319251 A1
(43) Pub. Date: Dec. 25, 2008

(54) Personal Electromagnetic Energy Dampener

(76) Inventor: John Mecca, Kings Park, NY (US)

Correspondence Address: JOHN MECCA, 119 WHITTIER DRIVE, KINGS PARKS, NY, 11754, US

(21) Appl. No.: 11/766,284

(22) Filed: Jun. 21, 2007

(51) Int. Cl. A61M 21/00

(52) U.S. Cl. 600/26

(57) ABSTRACT

The invention relates to placement of resistive bands around the appendages of a living body dampening any electromagnetic energy pervading such a body from internal or external sources by reactive induction into the band. and that the band be of resistive material, causes such energy to be absorbed and dissipated generally through Brownian motion of the particles of carbon for example embedded in the bands. To increase the IQ (intelligence quotient) and tissue health and normalcy by reduction of electromagnetic energy on the living tissue.

### BACKGROUND OF INVENTION

Personal energy reduction techniques have previously been the use of suits made of metallic material with properties to reduce the energy impacting the living body. The consideration of reducing the energy on and in the body is not easily addressed in previous art where the basis of there operation is preventative coupling into the body. Were also the use of such metallic material also behaves in a contradictory way and acts as an antenna where the energy thereby coalescing on the metallic material contradicts the purpose of controlling such energy from materially running up and down the living body's tissue causing various unhealthy effects. Environmental electromagnetic energy impinging on living tissue causes various effects by coupling to such tissue and those energies at specific frequencies affect various parts of the body according to frequency. At frequencies near the body's natural resonant frequency, RF energy is absorbed more efficiently, and maximum heating occurs. In adults, this frequency usually is about 35 MHz if the person is grounded, and about 70 MHz if the person's body is insulated from the ground. Also, body parts may be resonant; the adult head, for example is resonant around 400 MHz, while a baby's smaller head resonates near 700 Mhz. The advantages of this technique over other techniques is the real reduction of the effects of electromagnetic energy on the body and can be worn as independent bands, over or under traditional clothing and or on or inside metallic protective suits. The bands are able to moderate the resonance of the living tissue and dampen the effect of resonance and hence the frequency, electric and magnetic components of radio frequency energy.

### SUMMARY OF INVENTION

The circuit of a band is where the material arranged in a band configuration around a living being's appendage thereby does reduce the deleterious effects of electromagnetic energy on the living being; this action accomplished by the band being composed of material that is resistive. Such material composition is generally permeated by carbon particles. Such action of depletion by absorption of the electromagnetic energy is accomplished by the induction of the DPE rings, where such rings act as electronic chokes similar and the same as when a ring of conductive or less conductive material known as a resistive material surrounds in rig like fashion a conductor having energy coalescing on and inside it. Such reaction to reduce the free flow of energy on a current carrying conductor has to do with a subsequent induction by that flow of current to induce a current in the rings EED's where such induced current in the PDE has an effect on the current in the conductor pertinent to the magnetic, electric and frequency associated with such electromagnetic energy coalescing upon it. The effect of the ring EED has as a dampening effect is much like an electronic choke in the form of an insulated wire winding around a conductor. The dampening of electric, magnetic and frequency associated energies on and in the living tissue occurs as a result of the counter electromagnetic reaction from the corresponding complimentary field induced in the rings (EED's); whereupon that the current in the living tissue is as a conductor along with its associated energies. The dynamic energy in and around the living tissue will then be opposed by the rings EED's quasi static field being previously a complimentary field to the living tissue as a conductor. Each time the dynamic energies from the living tissue resonate into an opposite direction they will be opposed and therefore dampened by the EED bands quasi static field. The resistance of such bands will vary according to the environmental requirements. In strong electric fields commonly existing in the environment as an example 5-20 mega ohms determined with probes touching the band material placed one half inch apart is a good approximate value.

### BRIEF DESCRIPTION OF THE DRAWINGS

FIG. 1 generally a single band EED and associated specifics.

FIG. 2 generally a single band with addition of a buckle to connect the two ends of the single band shown. Such bands with a break can have a means to connect them simply additional length to allow the two ends to be overlapped and tied and knotted as a means to connect those ends of the ban around the living tissue.

FIG. 3 where (a) shows a cross section of a single band, where (b) shows the position of a conductor running the entire length of the resistive material encapsulating it and a second conductor on the outside also running the entire length of the resistive material. Both conductors have the option of being shorter than the length of the resistive band and are not required to go the entire length of the resistive band and can also be segmented so that there are for example four conductors in axial alignment within or on the outside of the resistive material that do not touch one another and do not therefore comprise a single conductor; lastly (c) showing perforations through the band and inference of intaglio where such optional geometries in alteration of a bands mass, shape of the holes and surface deformations presented where they can and may have desirous effects upon the induced field effect discussed and subsequent beneficial health effects mentioned by employing such alterations.

FIG. 4 is the placement of EED's at various points of the body.

FIG. 5 incorporation of the EED's into hat shirt and pants.

FIG. 6 integration of EED's into clothing, attachment and integral insertion of EED material into the fabric.

FIG. 7 laminate glue application of EED into fabric.

FIG. 8 Common belt buckle as generic illustration of connection parts as are shown in the bands, implying any means for coupling the ends of the bands together.

FIG. 9 EED resistive material where it is used as full body coverage in the way of a suit.

### DETAILED DESCRIPTION OF THE PREFERRED EMBODIMENT

The EED drawings are of a preferred embodiment illustrating the variety of ways they can be arranged. Where FIG. 1 (1) is of a single band being generally flat and continuous, such bands can be elastic so that they need not have any buckle or means to connect the ends as they are continuous and without break. As well the band can be made long enough so as to be able to tie the ends together around living tissue by any one of a number of knots see FIG. 1 (1-a). As well the bands can be sewn onto or into elastic bands of cloth fabric or other such material.

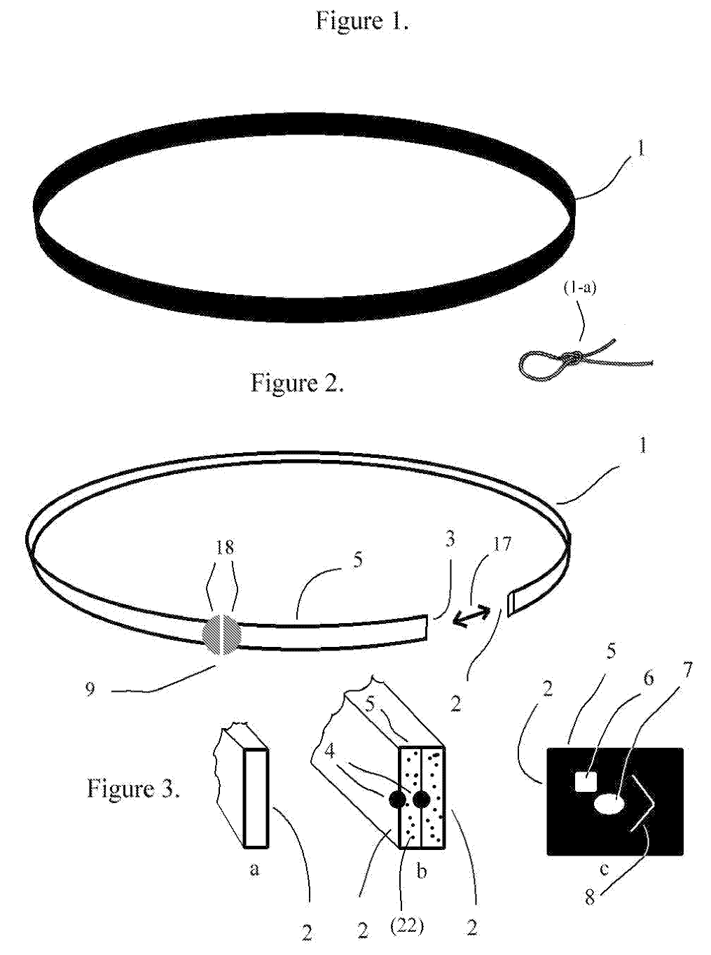

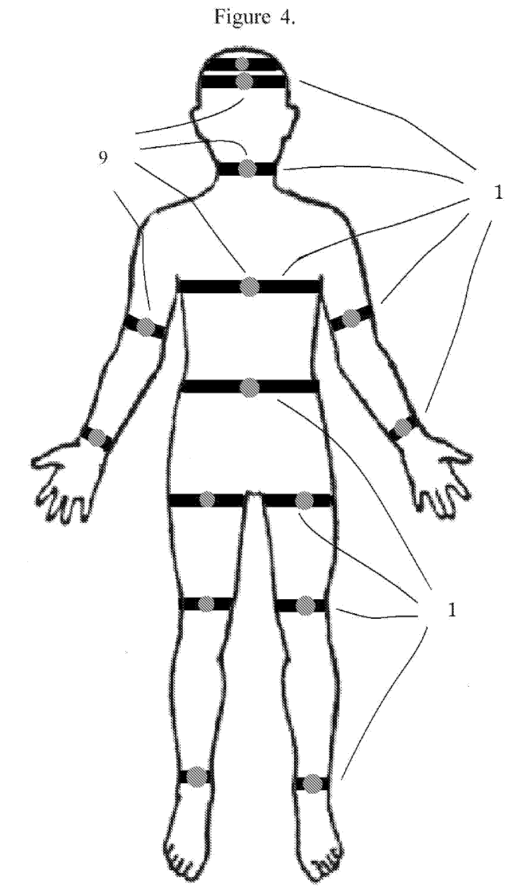

FIG. 2 being the same as FIG. 1 with the addition of designation of a fastener 9 of two ends of the one band, where the two ends of the band 18 couple together in the form of a common belt buckle, snap, zipper, Velcro or any other connection technique to link the ends together temporarily. Where 2 and 3 are the ends of the band in a cutaway of that band showing 17 as the arrow denoting the space where the band 1 is divided. FIG. 3 (a) is a cross section of the area of the end of the band seen as 2 in FIG. 2. FIG. 3 (b) is also an end view similar to FIG. 3(a) where the resistive material has particles 22 as an option of choice entrained within bands 1 such entrained particles can be carbon or metallic particles and or elastic bands 2 encapsulate a single or plurality of strands of conductive wire 4 made of any metal such as copper, stainless steel and or more conductive material specifically even resistive material as described in this work that is merely less resistive than the encapsulating material. Such wire 4 runs along the entire length of each band or as desired where it terminates. This center conductor increases the coalescing of electromagnetic energy onto itself by virtue of its greater conductance and where encapsulated acts to have the greater energy it has coalesces on it dampened by that surrounding material of greater resistive characteristics. FIG. 3 c has a side view of band 2 where 5 is a reference to the split division between two bands encompassing the center conductor 4. The numbers FIG. 3 c (6,7,8) are a variety of holes through or partially into the surface of the band for moderation of the effects the bands can have upon the fields they dampen, the effects such holes or depressions can have on the fields the bands encounter from energy in the environment and or in and on the living tissue the bands encapsulate will vary according the electrodynamic factors of how such fields interact. All bands can be in multiples in the same area and there can be independent bands laminated together isolated so that they do not resistively or conductively connect together; this plurality of bands is done where a multiplied effect of the fields mentioned is desired. All and any bands can be coated with a fabric or insulating material and have one band layered over another band as desired for moderation of the electromagnetic energy.

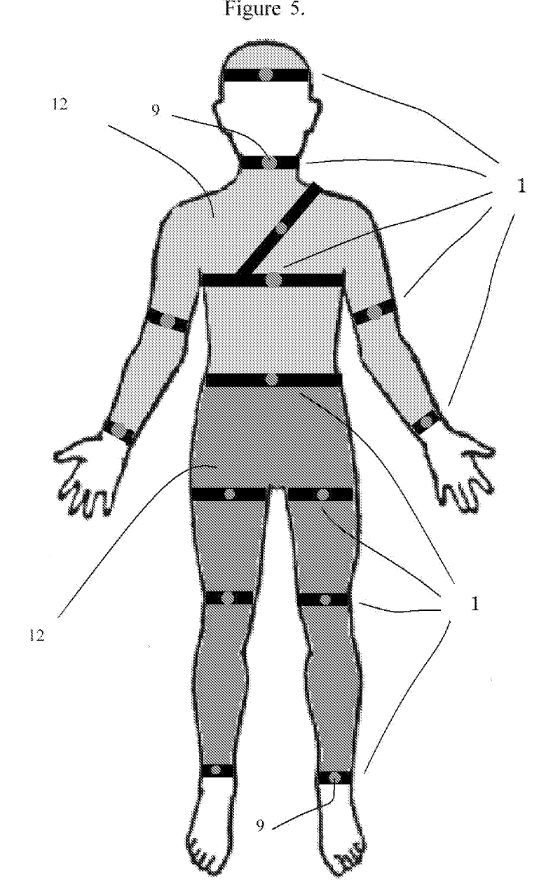

FIG. 4 shows the human form where bands 1 are wrapped around various parts of that form and buckles 9 for connecting the ends of individual bands together forming a continuous band around any part of the body. Also see the bands on the forehead of the human figure indicating that such bands can be in multiples in the same area.

FIG. 5 shows where bands 1 EED's can be incorporated into clothing of hat, shirt, pants shown numerically as 12's. Where buckles 9 as needed irrespective of need where in lieu of buckles 9 elastic material can be utilized dispensing with buckles 9 generally. Bands can be vertically placed so as to dampen fields that follow differing directions across the living tissue as can be seen regarding the generally vertical placement of a band from the shoulder down to the chest.

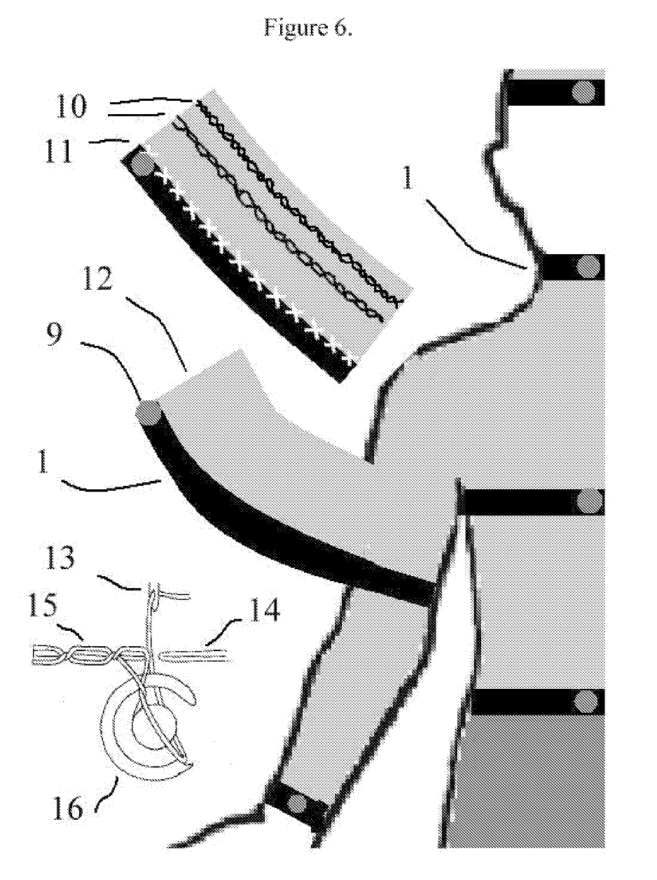

FIG. 6 shows the human form partly having on it bands 1 where a swatch of the clothing 12 is peeled away for observation having upon one edge a band 1 buckle 9 where further is shown stitching of a type of band applied by stitching into the material 12 band 1 resistive material 10 in a chain like stitch pattern and 11 as a method of cross stitches holding the resistive material in place inside or outside the clothing fabric to attach such resistive material where necessary; the means of attachment illustrated by sewing machine bobbin with needle commonly understood as 13,14,15 and 16 where the thread 15 being used to alternately attach an EED band to clothing or the thread itself being EED resistive material sewn into the garment as is seen by chain stitch 10. Also a plurality of bands 10 can be incorporated one next to each other with the use of the EED material as the thread like material incorporated.

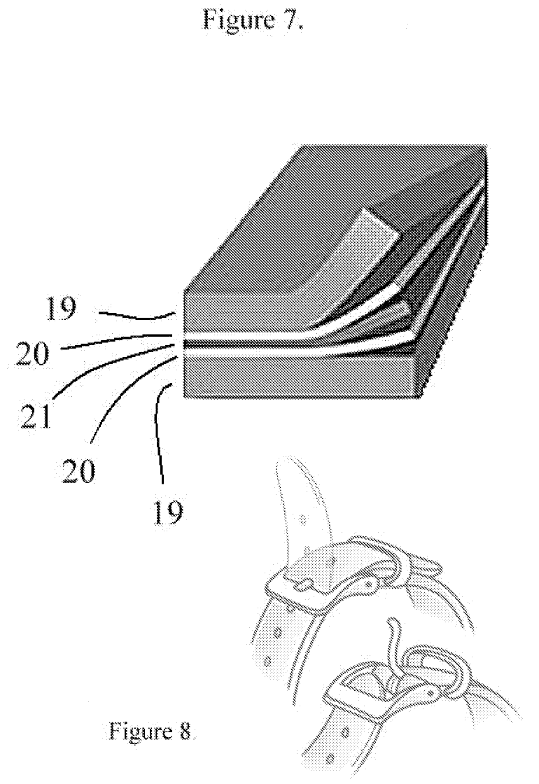

FIG. 7 shows as desired EED resistive material 21 sandwiched in between optional fabric 19 and held permanently there by adhesive 20's. The use of adhesive to maintain the position of the EED material in place on the inside and outside of clothing being the primary objective by the FIG. 5; as well as for adhesive technique for attachment to clothing after a such clothing was manufactured as an afterthought to add to such clothes.

FIG. 8 being a common belt buckle variously represented as one of any means to couple one free end to another of a band temporarily as described such as the preferred embodiments number 9 and the two separate of its ends 18 that couple together forming buckle 9.

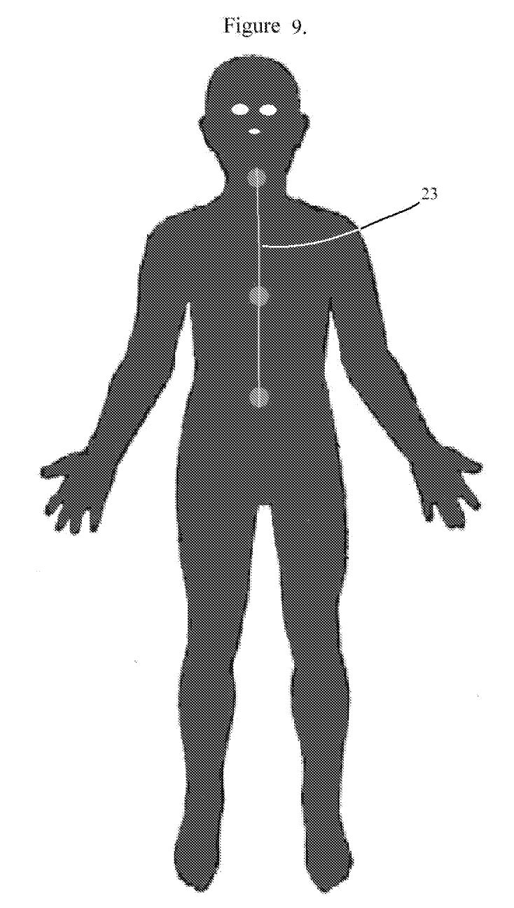

FIG. 9 is a complete body suit where the principal reference is that it is generally a single band encapsulating the body for the same purposes as a single band with the added issue of overall dampening and depreciating of the deleterious effects of electromagnetic energy on the living tissue; FIG. 29 is a vertical line representative of a split in the suit for the person to enter it, the small gray discs are the buckle connectors in this FIG. 9 to hold closed the split in the suit used for entry of a person; closure methods can include any mean such as zipper, Velcro, buttons or other methods. Note on head of figure three holes corresponding to the eyes and nose can be openings for seeing and breathing.

While the invention herein disclosed has been described by means of specific embodiments, numerous modifications and variations could be made thereto by those skilled in the art without departing from the scope and spirit of the invention set forth in the claims.

What is claimed is:

1. A single resistive band or plurality of such bands being flexible elastic resistive material applied in a circuitous fashion where one end of such band communicates to the other end forming a circuitous continuous band around living tissue; where said band being resistive by nature of its composition of flexible elastic rubber like material being impregnated with carbon or other semi-conductive material.

2. The band of claim 1 where the circuitous continuous band around living tissue is complete and without break requiring no coupling m mechanism to cause its application to the living tissue.

3. The band of claim one where the length of the band has two ends terminating in a mechanism to temporarily cause the two ends to connect such as a belt buckle, velcro, or other means not excluding a simple knot

4. Band of claim 1 where it's resistive nature has added to it additional particles of a more conductive material than that of claim 1, such particles are distributed throughout the length and width of the band of claim 1, such particles can be of different conductive grade or density of carbon particles or made of any conductors or metal particle as well as organized matter in the form of particles composed of nanomaterials and or as well as organized matter in the form of particles composed of metamaterials.

5. Band of claim 1 where said resistive band has a single or plural conductor running throughout its length and or on the surface of band 1.

6. Band of claim 1 can be woven into a substrate such as cloth or affixed using adhesive means to cause permanent attachment onto clothing or inside clothing.

7. The claim of 1 where geometric holes in said band either where such holes go from the inside of the band to the outside or only partially into the surface of the band as means to moderate environmental field density surrounding the band by the lack of mass or indentation or shape of the indentation or hole.

8. Where the claim of 1 being a resistive band is further a full body suit as a single band around the living tissue.

9. The claim 9 wherein bands of claim 1 can be affixed circuitously around the various living tissue appendages including the torso, such bands can be on the inside or outside of such full body suit or integrally laminated inserted within the suits resistive material thickness.
